(19) United States
(12) Patent Application Publication
(10) Pub. No.: US 2022/0030985 A1
(43) Pub. Date: Feb. 3, 2022

(54) SOFT ACTUATOR AND METHOD OF MAKING THE SAME

(71) Applicant: The Regents of the University of California, Oakland, CA (US)

(72) Inventors: Yon VISELL, Santa Barbara, CA (US); Elliot HAWKES, Goleta, CA (US); Do Thanh NHO, Goleta, CA (US); Mengjia ZHU, Goleta, CA (US)

(21) Appl. No.: 17/275,731

(22) PCT Filed: Sep. 13, 2019
(86) PCT No.: PCT/US2019/051032

§ 371 Date: Mar. 12, 2021

Related U.S. Application Data
(60) Provisional application No. 62/731,583, filed on Sep. 14, 2018.

(51) Int. Cl. A41D 19/00; G08B 6/00; A43B 3/00; A41D 1/00

(52) U.S. Cl. CPC A41D 19/0024; A41D 1/002; A43B 3/0005; G08B 6/00

(57) ABSTRACT

A selectively actuated textile includes one or more pieces of fabric having one or more circumferentially constrained channels and one or more hollow elastic tubes located within the circumferentially constrained channels and configured to receive a working fluid. Selectively providing or removing working fluid from the hollow elastic tubes provides for selective actuation of the textile.

### CROSS-REFERENCE TO RELATED APPLICATIONS

This application claims the benefit of U.S. Provisional Application No. 62/731,583, filed Sep. 14, 2018, titled “SOFT ACTUATOR AND METHOD OF MAKING THE SAME”. The provisional application is incorporated by reference in its entirety.

### GOVERNMENT LICENSE RIGHTS

This invention was made with government support under contracts 1623459 and 1628831 awarded by the National Science Foundation. The government has certain rights in the invention.

### FIELD OF THE INVENTION

The present invention is related to actuators, and in particular to soft actuators.

### BACKGROUND

An array of soft actuator technologies has emerged in recent years, offering the promise to enable safe and powerful actuation in a growing number of common environments and applications, and facilitating new designs for soft robotic systems. Among these actuators are artificial muscles, which emulate the ability of biological muscles to perform mechanical work via contractile motions. Many approaches to the design of such actuators have been proposed, for applications in industry, wearable devices, or medical systems.

Typical actuators utilized in robotic applications—such as electromagnetic motors, hydraulic pistons, and piezoelectric actuators, are inappropriate due to the rigidity associated with these systems. A variety of ‘soft’ actuators have been proposed, including shape memory alloys (SMA), shape memory polymers (SMP), electroactive polymers (EAPs), and pneumatic artificial muscles (PAM) such as the McKibben muscle, which can be selectively actuated to provide a force and embedded within garments and textiles. However, each of the proposed soft actuators has drawbacks. For example, SMA provides high force density, but poor total strain (i.e., displacement or movement) as well as poor efficiency. In addition, SMAs require heat to actuate, which makes it a poor choice for embedding in textiles/fabrics. SMPs exhibit very high strains, but only limited force production. EAPs provide large strains but require high voltages for operation. Pneumatic artificial muscles provide a high force density, but relatively poor total strain.

It would therefore be beneficial to develop a soft actuator that overcomes these deficiencies, providing high force density in combination with high strain.

### SUMMARY OF THE INVENTION

According to one aspect, a method of fabricating a soft actuator includes forming one or more channels within one or more pieces of fabric, wherein the channels provide constraint in a circumferential direction. One or more hollow elastic tubes are placed within the one or more channels. At least one end of each of the one or more hollow elastic tubes is connected to a delivery system capable of providing a working fluid to the one or more hollow elastic tubes.

According to another aspect, a selectively actuated textile is provided that includes one or more pieces of fabric having one or more circumferentially constrained channels formed within the one or more pieces of fabric. In addition, the selectively actuated textile includes one or more hollow elastic tubes positioned within the one or more channels and configured to receive a working fluid, wherein working fluid is selectively provided to or removed from the one or more hollow elastic tubes to actuate the selectively actuated textile.

According to another aspect, a haptic feedback garment is provided that includes one or more soft actuators, one or more tubes, and one or more pumps. The one or more soft actuators are embedded within the haptic feedback garment, wherein each actuator includes a hollow elastic tube configured to receive a working fluid. Each of the one or more tubes is connected on a first end to an input associated with each of the soft actuators. Each of the one or more pumps is configured to provide a working fluid to one of the one or more soft actuators via one of the one or more tubes, wherein application of the working fluid to the soft actuator provides a haptic response to a user wearing the haptic feedback garment.

### BRIEF DESCRIPTION OF THE DRAWINGS

FIGS. 1A and 1B are side and cross-sectional schematics of a soft actuator in a first, pressurized state according to some embodiments.

FIGS. 1C and 1D are side and cross-sectional schematics of a soft actuator in a second, non-pressurized or relaxed state according to some embodiments.

FIGS. 2A and 2B are cross-sectional views of a soft actuator that illustrates the forces applied within the textile according to some embodiments.

FIG. 3 is a schematic illustrating the fabrication of a soft actuator according to some embodiments.

FIGS. 4A-4B are schematic views of a soft actuator operating in a force mode according to some embodiments.

FIGS. 4C-4E are views illustrating a plurality of applications utilizing soft actuators operating in a force mode according to some embodiments.

FIGS. 5A-5B are schematic views of a soft actuator operating in a displacement mode according to some embodiments.

FIGS. 5C-5D are views illustrating a soft actuator operating in a displacement mode according to some embodiments.

FIGS. 6A-6D are graphs illustrating the relationship between force and displacement, length and volume, force and displacement, and external force and displacement according to some embodiments.

FIGS. 7A-7C are graphs illustrating the relationship between force and displacement according to some embodiments.

FIGS. 7D-7F are graphs illustrating the results of tests according to some embodiments.

FIGS. 8A and 8B are schematic views of a soft actuator that incorporates one or more soft sensors according to some embodiments.

FIGS. 9A and 9B are photographs of soft actuator that incorporates one or more soft sensors according to some embodiments.

FIG. 10 is a picture of a glove that utilizes soft actuators and soft sensors according to some embodiments.

FIGS. 11a and 11b are side views of a multi-actuated Fluidic Fabric Muscle Sheet (FFMS) according to some embodiments.

FIGS. 12a-12c are views of a Fluidic Fabric Muscle Sheet (FFMS) combined with a second, passive layer to provide out-of-plane bending according to some embodiments.

FIGS. 13a-13e are views of a Fluidic Fabric Muscle Sheet (FFMS) that provides biaxial bending according to some embodiments.

### DETAILED DESCRIPTION

The present disclosure is directed generally to a soft actuator that is actuated in response to a change in volume and/or pressure of a working fluid. In general, the soft actuator includes one or more hollow elastic fibers located within a textile fabric, wherein the hollow elastic fibers are constrained circumferentially. Actuation of the device includes selectively providing a working fluid into the one or more hollow elastic fibers to operate the actuator in either a pressurized state or a relaxed state. In a pressurized state, the circumferential constraints placed around the hollow elastic fibers prevent the hollow elastic fibers from expanding in a circumferential direction, resulting in the actuator lengthening in a direction transverse to the circumferential direction (i.e., along the axis of the hollow elastic fibers). When lengthened, elastic energy is stored in the elastic fibers of the soft actuator. When operated in a relaxed state by removing the working fluid the stored elastic energy causes the soft actuator to contract.

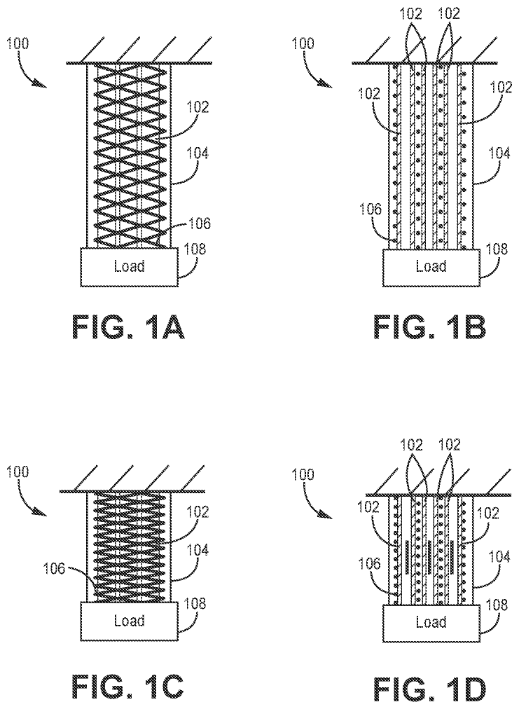

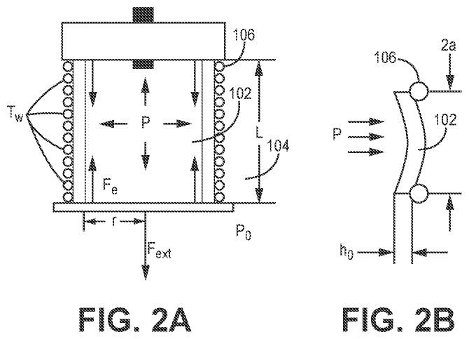

FIGS. 1A and 1B are side and cross-sectional schematics, respectively, of a soft actuator 100 in a first, pressurized state according to some embodiments, and FIGS. 1C and 1D are side and cross-sectional schematics, respectively, of a soft actuator 100 in a second, non-pressurized state according to some embodiments.

In the embodiment shown in FIGS. 1A-1D, a soft actuator 100 includes one or more hollow elastic fibers 102, fabric 104, and circumferential constraint 106. In the cross-sectional view shown in FIG. 1B, a plurality of hollow elastic fibers 102 extend parallel to one another along the length of elastic fabric 104. In some embodiments, the plurality of hollow elastic fibers 102 may include a single elastic fiber 102 that is wound back and forth to provide the plurality of hollow elastic fibers 102 visible in FIGS. 1A-1D. The one or more hollow elastic fibers 102 are configured for coupling to a hydraulic or pneumatic motor that supplies a working fluid (either hydraulic or pneumatic) to the one or more hollow elastic fibers 102.

In the embodiment shown in FIGS. 1A-1D, the plurality of hollow elastic fibers 102 are restrained circumferentially by circumferential constraint 106. In some embodiments, circumferential constraint 106 is comprised of inextensible stitching that extends along the outer circumference of the hollow elastic fiber. That is, in some embodiments the stitching defines a channel in which the hollow elastic fibers are housed. In some embodiments, the stitching is directed linearly along the respective sides of the channel in what is known as a side-stitch pattern. In other embodiments, the stitching is arranged in a different pattern, such as cross-stitched back and forth over the channel such that the inextensible stitching extends in a direction approximately transverse to the direction of actuation of the hollow elastic fiber 102 as shown in FIGS. 1A-1D. The cross-sectional view shown in FIG. 1B illustrates how the stitching 106 surrounds and constrains circumferentially each hollow elastic fiber 102. Although the embodiment shown in FIGS. 1A-1D utilizes inextensible stitching 106 to restrain hollow elastic fibers 102, in other embodiments other means may be utilized to provide circumferential restraint of each of the one or more hollow elastic fibers 102. For example, in some embodiments the one or more fabric layers 104 are glued to form the circumferential constraint 106. In other embodiments heat sealing and/or ultrasonic welding to form the circumferential constraint 106.

As described above, hollow elastic fibers 104 are configured to be coupled at a first end (e.g., top end) to a hydraulic or pneumatic pump that provides a working fluid to the one or more hollow elastic fibers 104. In some embodiments, the working fluid is nearly incompressible (e.g., liquid or hydraulic working fluid) and in other embodiments may be compressible (e.g., gaseous or pneumatic working fluid). In some embodiments, a pump is utilized to provide the working fluid to the one or more hollow elastic fibers 102. The working fluid may be gaseous or liquid, and therefore the pump utilized may be pneumatic or hydraulic. For example, in some embodiments a syringe and linear motor is utilized to provide the working fluid (e.g., liquid) to the one or more hollow elastic fibers 102, resulting in the length of the soft actuator 100 to increase.

In the embodiment shown in FIGS. 1A and 1B, the selectively-actuated textile 100 is in a first, pressurized state in which a pressurized working fluid is provided to each of the hollow elastic fibers 102. Because the one or more hollow elastic fibers 102 are constrained in the circumferential direction (i.e., the x-direction) by circumferential constraint 106, the only direction in which the one or more hollow elastic fiber 102 is allowed to extend in response to the pressurized working fluid is in the longitudinal direction (i.e., the y-direction). More particularly, the provision of pressurized working fluid causes the volume of hollow elastic fiber 102 to increase. Because of the circumferential constraint, the one or more hollow elastic fibers 102 lengthens to accommodate the increase in volume. As the hollow elastic fiber 102 lengthens, the entire textile (e.g., fabric 104) lengthens. Lengthening of the fabric 104 and hollow elastic fibers 102 stores potential energy in the elastic components of the selectively-actuated textile 100. When the pressure associated with the working fluid is removed, the one or more hollow elastic fibers 102 experience a decrease in volume, which causes both the hollow elastic fibers 102 and the elastic fabric 104 to release stored elastic energy, allowing the textile to apply a contracting force to load 108 as shown in FIGS. 1C and 1D.

A variety of different materials may be utilized to fabricate the selectively actuated textile 100. The selection of materials may depend on the application, specifically on the elongation and/or force requirements. In some embodiments, the hollow elastic fibers 102 must be elastic. The fabric 104 may be elastic or inelastic, but the dynamic forces delivered by the actuator depend, in part, on the tension remaining in the hollow elastic fibers 102 and/or the elastic fabric (if present) 104 after the addition of the hydraulic fluid. In some embodiments, the fabric is selected to provide negligible stiffness in the axial direction of the tube. In some embodiments, non-stretchable fabrics such as cotton weaves are utilized in combination with a wrinkling process to allow lengthening in the axial direction. In some embodiments, stretchable fabrics may be utilized in combination with across-tube stitching to provide radial constrain of the hollow elastic fibers 102. Stretchable fabrics may include uniaxially elastic (two-way stretch) or biaxially elastic (four-way stretch). In some embodiments, this may include elastic fibers such as Spandex, spun into stretchable yarn, and integrated along weft, warp, or both directions of the weave, yielding one or two-way stretch fabric, respectively. In other embodiments, either elastic or non-elastic fibers may be used to create a knit, wherein stretchability depends on the design of the looping structure. In some embodiments, the stiffness of the elastic fabric 104 and the hollow elastic fibers 102 is approximately equal. In other embodiments, the fabric 104 is inelastic but is bunched in a manner that makes it less stiff than the hollow elastic fibers 102. The stitching 106 circumferentially constrains the hollow elastic fibers 102 but allows for lengthening of the overall textile 100. In some embodiments, stitching 106 is provided transverse to the direction of actuation (i.e., lengthening direction), in a cross-stitch configuration. In some embodiments, stitching 106 defines the width of channels utilized to accept hollow elastic fibers 102, and the fabric is used to provide the circumferential constraint. In some embodiments, the width of the channel defined by the stitching 106 and the diameter of the hollow elastic fibers 102 are selected such that the increase in fluid volume in the hollow elastic fiber 102 elicits the greatest increase in the rest length of the rest length of the hollow elastic fiber 102. In some embodiments, the width of the channel defined by the stitches 106 are equal to approximately one-half of the outer circumference of the hollow elastic fiber 102. In some embodiments, the type of thread utilized for the stitches may include inextensible high-strength nylon thread

In addition to selection of materials for hollow elastic fibers 102, elastic fabric 104 and circumferential constraint 106, various fluids may be utilized with respect to hollow elastic fibers 102. In some embodiments, the working fluid is an incompressible working fluid (e.g., liquid). One benefit of incompressible fluids is that the lengthening of the textile is directly related to the volume of incompressible fluid provided, which provides quasi-static response. In other embodiments, the working fluid may be a compressible fluid (e.g., gas). Benefits of compressible fluids include a reduction in mass of the actuator (as compared with embodiments that utilized incompressible fluids). However, the volume-pressure relationship are not static, and therefore complicate methods of controlling the length of the textile in response to a measured attribute (e.g., pressure).

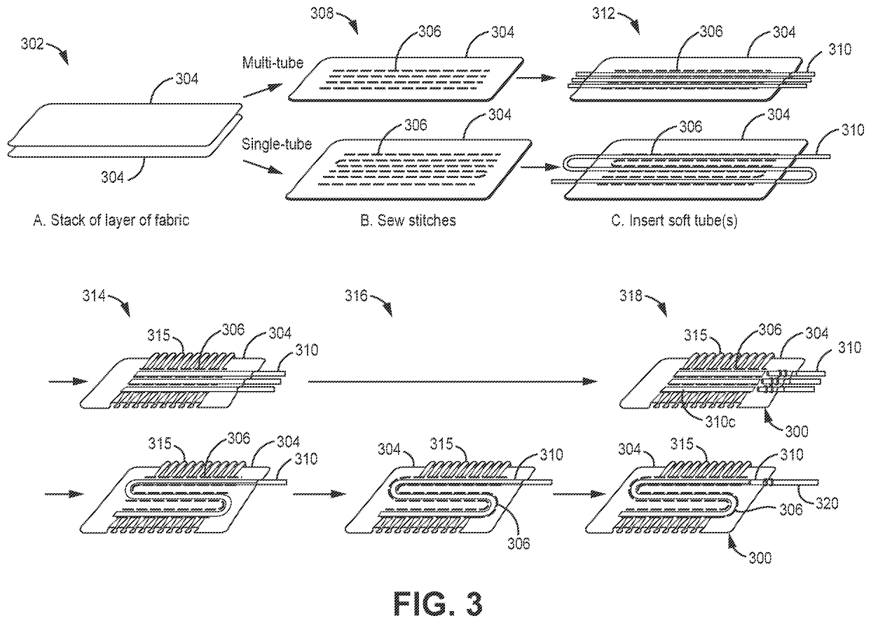

FIGS. 2A and 2B are side schematic views of a selectively actuated textile 100 that illustrates the forces acting on a single hollow elastic fiber 102 according to some embodiments. In the embodiment shown in FIGS. 2A and 2B, hollow elastic fiber 102 is constrained circumferentially by inelastic stitches 106.

In the circumferential direction, the tension T_{w} in the stitches 106 wrapped around the hollow elastic fiber 102 balances the force of pressure P exerted by the working fluid within the hollow elastic fiber 102, which can be expressed as, T_{w}N=2PLr, where N is the number of wraps or stitches, L is the current length of the hollow elastic fiber 102, and r is the radius of the hollow elastic fiber 102. In the radial direction, the stress in the hollow elastic fiber 102 is relatively small (equal to P on the inner surface), but between fibers, the tubing can bulge as shown in FIG. 2B. The deflection of the center of the bulge away from the neutral position is approximated for large stretch as

$$δ = a tan ( \frac{β}{2} )$$

wherein

$$β = \frac{a P}{μ {h}_{0}} ,$$

and a is half the separation of the fibers, μ is the shear modulus, and h₀ is the thickness with no pressure. This model suggests that a thicker wall, lower operating pressures, and a denser stitching all aid in the prevention of ballooning. However, when the fabric is a non-stretch fabric, the fabric itself prevents any ballooning.

In the axial direction, the elastic force in the hollow elastic fiber, Fₑ, that causes the elastic fiber 102 to contract is balanced by the external force, Fₑₓₜ and the force due to the pressure inside the fiber, Fₚ, wherein Fₑₓₜ=Fₑ−F, where Fₑ=k(L−L_{f}) and Fₚ=AP: Here, k is the stiffness of the elastic tubing, L_{f} is the free length of the elastic membrane, and A is the cross-sectional area of the tubing (i.e., πr²). This model suggests a thicker wall can increase the elastic force Fₑ (by increasing the stiffness coefficient k), but also that this thicker wall requires a higher pressure to attain elastic force Fₑ=0. A number of simplifications were made in describing the model: rubber stress-strain is considered linear, Poisson contraction of the wall is ignored, and angle changes of the stitching are considered small.

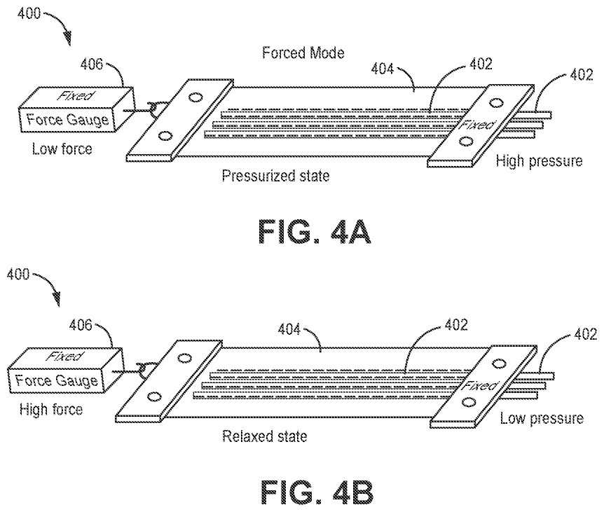

FIG. 3 is diagram illustrating fabrication of a soft actuator 300 according to some embodiments. At step 302, two or more pieces of fabric 304 are stacked together. As described in more detail below, fabric 304 may be non-stretch, a two-way stretch fabric, or a four-way stretch fabric. At step 308, one or more channels are formed between the stacked layers of fabric 304 that form a circumferential constraint around the channels. In some embodiments, inextensible stitching 306 is utilized to form these circumferential constraints. Various types of stitches may be utilized, including side-stitching, cross-stitching, and/or a combination of side-stitching and cross-stitching.

In some embodiments, the channels formed by way of stitching include a plurality of openings for receiving a plurality of individual hollow elastic fibers 310, as shown in the top embodiment of steps 308 and 312. In other embodiments, the channels formed by way of stitching include openings for receiving a single hollow elastic fiber 310 which is wound back and forth as shown in the bottom embodiment of steps 308 and 312. Various materials may be utilized for hollow elastic fiber 310, and selection may be based on attributes such as desired lengthening, force generation, and Young's modulus. For example, in some embodiments, latex tubing generates a relatively large elastic force, has a relatively large Young's modulus, and is capable of extending up to five times its original size. In other embodiments, silicone tubing may be utilized.

At step 314, one end of the hollow elastic fibers 310 are sealed. In the embodiment in which a plurality of individual hollow elastic fibers 310 are utilized, this may include sealing each of the hollow elastic fibers 310 on one side of the fabric (i.e., left side). The other end of the hollow elastic fibers 310 remains open for connection to the pneumatic or hydraulic pump for receiving the working fluid during actuation. In embodiments in which a single hollow elastic fiber 310 is utilized, this requires that one end of the fiber 310 be sealed.

In some embodiments, at step 314 the fabric 304 may be wrinkled (if necessary) to allow the fabric to lengthen during actuation. In some embodiments, wrinkling of fabric 304 may be utilized to allow fabric 304 to lengthen, by taking up the slack afforded by the wrinkles. This may be particularly beneficial if utilizing a non-stretch or inelastic fabric. In other embodiments, wrinkling of the fabric 304 may not be required. In particular, if fabric 304 is a stretch or elastic fabric, it may not be necessary to wrinkle the fabric as shown at step 314. In the embodiment shown at step 314, wrinkles 315 are shown. In some embodiments, wrinkling of the fabric includes stretching the hollow elastic fibers 310 along the axial direction, clamping or otherwise securing the fibers 310 to maintain the fibers in an extended state, bunching/wrinkling the fabric 304, and then attaching the hollow elastic fibers 310 to the fabric 304 at the ends of the actuator 300 to ensure a pre-tensioning of the hollow elastic fibers 310. In some embodiments, because the fabric 304 is inelastic, side-stitching may be utilized to form the channels for receiving the hollow elastic fibers 310 because the inelastic nature of the fabric 304 will provide circumferential constraint.

At step 316, additional stitching may be provided as necessary following sealing of the hollow elastic fibers at step 314. For example, this may include adding cross-stitching to stitching that originally included only side-stitching, or vice versa. In the embodiment shown in the bottom, additional stitching is provided around the channels that switch back and forth.

At step 318, the one or more hollow elastic fibers 310 are connected to fittings and/or tubing 320 for delivery of the working fluid. For example, in some embodiments the one or more hollow elastic fibers 310 are connected to a rigid tube via one or more fittings that allow the working fluid to be selectively provided to the hollow elastic fibers 310.

Table 1, provided above, describes a number of the various features/attributes that may be selected with respect to fabrication of the planar actuation. For example, various fabrics are described in the left column, which includes non-stretch fabric, two-way stretch fabric, and four-way stretch fabric. In addition, pros and cons of each are provided. For example, non-stretch fabric is described as not requiring cross-stitching, and because the fabric does not stretch does not exhibit the ballooning problems noted with respect to FIG. 2B, above. However, non-stretch fabric is not stretchable, and therefore does not naturally allow for lengthening in the direction of actuation. In embodiments utilizing non-stretch fabric, wrinkling of the fabric is required to allow for the desired lengthening of the actuator. In comparison, two-way stretch fabric (allows stretching along one-axis) allows for lengthening of the planar actuator without wrinkling but requires alignment of the hollow elastic fibers along the direction of stretchability. Four-way stretch fabric, which allows stretching along both axes) allows lengthening of the planar actuator without wrinkling and does not require alignment of the hollow elastic tubes with the direction of stretchability but does require the use of cross-stitching (as opposed to just side-stitches) in order to prevent ballooning issues.

In addition, the top row of Table 1 describes various types of stitching that may be utilized, including side-stitches, cross-stitches, and a combination of side-stitches and cross-stitches. Pros and cons of each are provided. For example, a benefit of side-stitches is that the stitching process itself is relatively straightforward, and insertion of the hollow elastic tubes is fairly easy. However, side-stitching suffers from ballooning issues when utilizing stretchable fabrics. In contrast, cross-stitches provide better circumferential constraint of the hollow elastic fibers/tubing but is more difficult to form the channels for accepting the hollow elastic tubing. A combination of side-stitches and cross-stitches can provide improved performance with respect to reducing ballooning issues but is similarly difficult to form the channels for accepting the hollow elastic tubing.

In the embodiment shown in FIG. 3, the actuator 300 is a soft planar actuator. For example, the planar actuator is approximately two-dimensional (e.g., length and width), with a relatively small depth. In some embodiments, this is particularly beneficial for use in articles of clothing such as sleeves.

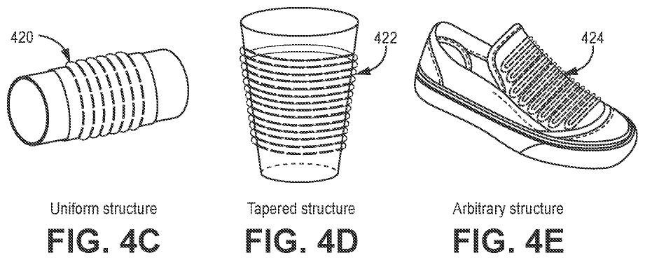

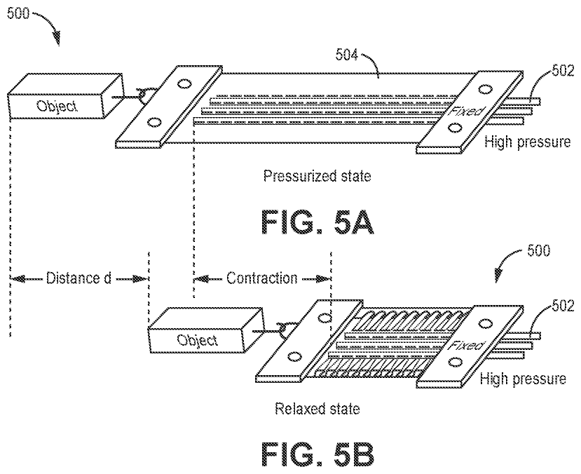

FIGS. 4A and 4B illustration a force mode planar actuator 400 operating in a pressurized state and a relaxed state, respectively. A force mode planar actuator is utilized to selectively generate a force on a desired load, and may be utilized in a number of applications, examples of some of which are shown in FIG. 4C.

In the embodiment shown in FIGS. 4A and 4B, planar actuator 400 includes a plurality of hollow elastic fibers 402 fabricated within a fabric 404. In this embodiment, both ends of the planar actuator 400 are fixed in space. Application of a working fluid to the hollow elastic fibers 402 opposes the elastic force Fₑ associated with the hollow elastic fibers 402 and/or fabric 404 and therefore reduces the force applied to the load 406 (i.e., fixed force gauge). That is, increasing the pressure of the working fluid provided to the planar actuator 400 reduces the force applied to the respective load. Conversely, reducing the pressure of the working fluid provided to the planar actuator 400 reduces the force provided by the working fluid to oppose the elastic force Fₑ, thereby increasing the force applied to the respective load.

The change in force provided by planar actuator 400 may be utilized in a number of applications. For example, compression applications in which the planar actuator is wrapped around a structure. FIGS. 4C-4E illustrates a plurality of such application, including a uniform structure 420, a tapered structure 422, or an arbitrarily shaped structure 424. The uniform structure 420 could be utilized to provide compression to a person's arm, while the tapered structure 422 may be more appropriate for providing compression on a patient's leg. The arbitrary structure 424 could be utilized on a variety of shapes, allowing force to be selectively applied via transition from a pressurized state to a relaxed state.

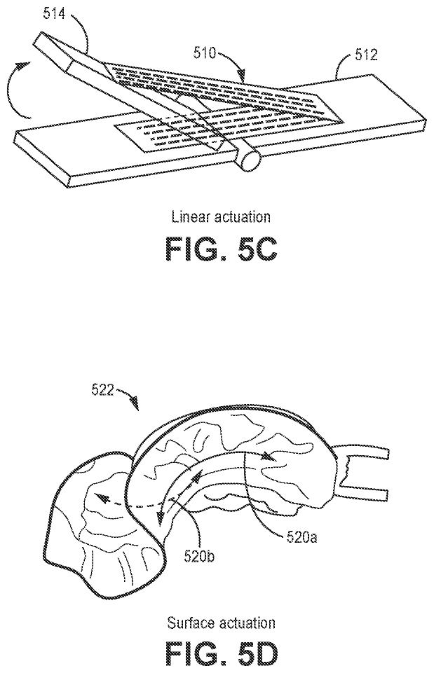

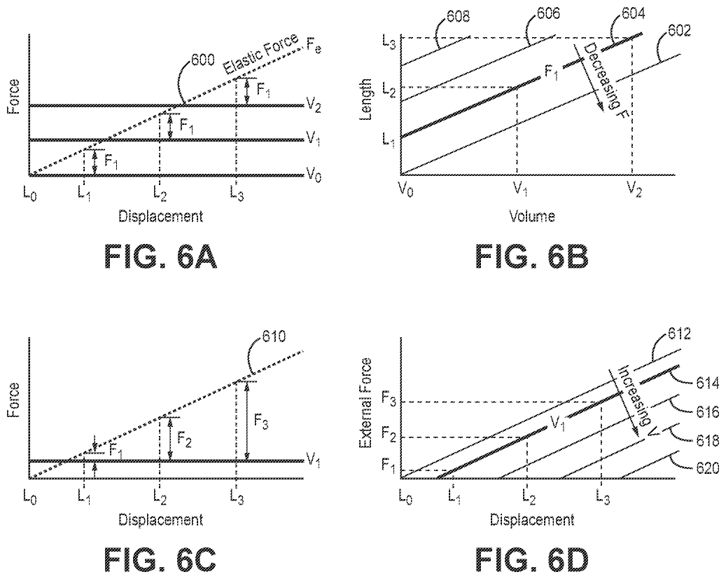

FIGS. 5A and 5B illustration a displacement mode planar actuator 500 operating in a pressurized state and a relaxed state, respectively. A displacement mode planar actuator 500 is utilized to selectively displace an object in a desired direction, and may be utilized in a number of applications, examples of some of which are shown in FIG. 5C.

In the embodiment shown in FIGS. 5A and 5B, planar actuator 500 includes a plurality of hollow elastic fibers 502 fabricated within a fabric 504. In this embodiment, one end of the planar actuator 500 is fixed in space, or neither end of the planar actuator 500 is fixed in space. Application of a working fluid to the hollow elastic fibers 502 opposes the elastic force Fₑ associated with the hollow elastic fibers 502 and/or fabric 504 and therefore causes the planar actuator 500 to lengthen or actuate in a desired direction as shown in FIG. 5A, in which planar actuator 500 is in a pressurized state. Reduction of the pressure of the working fluid causes the planar actuator 500 to contract in length in response to the elastic force Fₑ associated with the one or more hollow elastic fibers 502 and/or fabric 504. In the embodiment shown in FIGS. 5A and 5B, the total displacement of the object due to the contraction of the elastic fibers 502 is measured as a distance d. In some embodiments, such as that shown in FIG. 5B, fabric 502 is wrinkled to allow for the contraction and lengthening of the fabric 502 as desired.

The change in length provided by planar actuator 500 may be utilized in a number of applications. For example, planar actuator may be utilized in applications to lift a weight, operate a hinge structure (as shown in FIG. 5C) or to create surface tension (as shown in FIG. 5D). For example, in the linear application example, the planar actuator 510 is connected on a first end to a first portion 512 and on a second end to a second portion 514. The first and second portion are pivotable connected to one another. When in a pressurized state, the planar actuator 510 is lengthened, which allows the second portion 514 to pivot relative to the first portion 512 like a muscle relaxing. When in a relaxed state, the planar actuator 510 contracts, which causes the second portion to pivot upward relative to the first portion like a muscle contracting. In this way, the planar actuator 510 can be utilized to provide linear actuation.

In other application, the planar actuator may be utilized to provide surface actuation. In this embodiment, two sheet actuators 520a and 520b are combined vertically when both actuators are pressurized. Thus, the combined sheet 522 is flat when the pressure is high in both actuators 520a and 520b, while it generates biaxial curvature when both are at low pressure as shown in FIG. 5D.

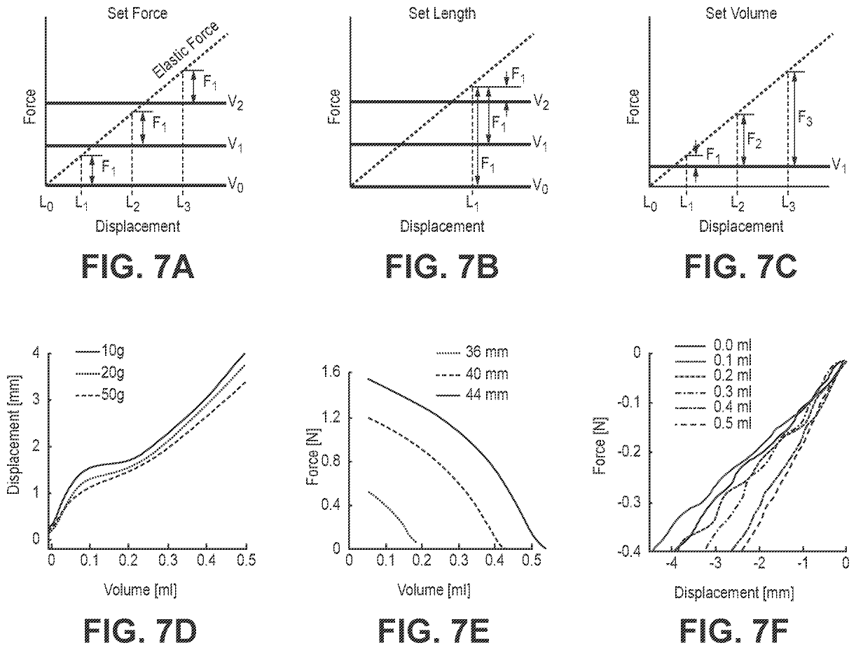

FIGS. 6A-6B are graphs illustrating the volume-displacement response of the planar actuator with a prescribed force (i.e., set force condition). In FIG. 6A, the elastic force generated in response to displacement in the direction of actuation is illustrated. As shown in FIG. 6A, displacement describes the lengthening of the actuator in response to an increase in pressure or volume of the working fluid. In the example provided, three discrete volumes of working fluid (V₀, V₁, and V₂) are provided. As the volume of working fluid increases, the elastic force generated by the planar actuator increases monotonically as shown in FIG. 6A

FIG. 6B illustrates the relationship between length and volume for a plurality of set force conditions (e.g., lines 602, 604, 606 and 608 represent set force levels F₀, F₁, F₂, and F₃). Once again, the length of the planar actuator is observed to increase monotonically in response to increases in the volume of the working fluid (incompressible fluid). This monotonically increasing relationship remains true despite the initial force exerted on the planar actuator.

FIGS. 6C and 6D illustrates the relationship between force and displacement assuming a constant volume. As shown in FIG. 6C, an increase in force results in an increase in displacement of the planar actuator (e.g., lengthening). For example, application of a force F₁ results in a displacement length of L₁. Assuming the volume of working fluid V₁ remains constant, an increase in force from F₁ to F₂ increases the displacement length from L₁ to L₂. Likewise, additional increases in force result in monotonical increases in displacement.

FIG. 6D illustrates the relationship between external force and displacement for a plurality of set volume conditions (e.g., lines 612, 614, 616, 618, and 620 represent set volumes V₀, V₁, V₂, V₃, and V₄). Displacement or lengthening of the planar actuator increases monotonically with increases in external force. For example, as external force increases from F₁ to F₂ to F₃ the measured displacement increases from length L₁ to L₂ to L₃, assuming volume of the working fluid remains unchanged. As shown in FIG. 6D, if the volume of working fluid is increased, the displacement curve is similarly increased.

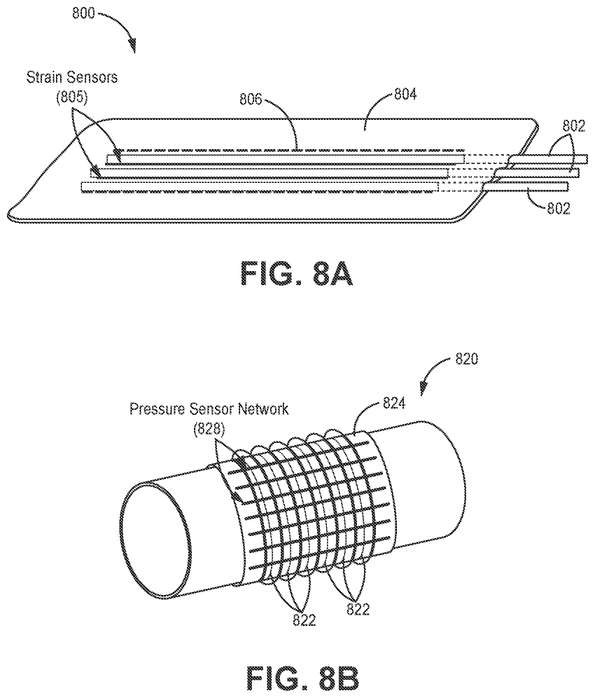

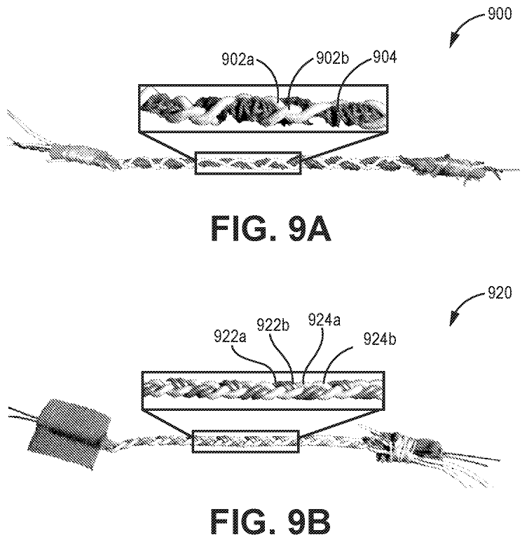

FIGS. 7A-7C are graphs illustrating the relationship between force and displacement assuming set force, set length and set volume, respectively. For example, FIG. 7A illustrates the relationship between force and displacement at various volumes of working fluid (i.e., incompressible working fluid). As the planar actuator increases in length (i.e., as displacement increases), the elastic force exerted by the planar actuator increases monotonically. That is, as the planar actuator is stretched or lengthened, the elastic force exerted by the planar actuator increases. In the graph shown in FIG. 7A, increases the volume of working fluid in the working actuator increases the length the actuator, assuming the external force F₁ applied to the actuator remains constant.

FIG. 7B illustrates the relationship between force and displacement given a set length L₁. Assuming a set length, the force applied by the planar actuator is related to the volume of the working fluid (incompressible working fluid). As the volume of the working fluid increases, the force exerted on an object increases (assuming that the length L₁ of the actuator remains constant).

FIG. 7C illustrates the relationship between force and displacement given a set volume V₁. In particular, assuming volume of the working fluid remains constant, increases in external force applied to the planar actuator result in an increase in displacement of the planar actuator. For example, increases in force from F₁ to F₂ to F₃, the displacement or length of the planar actuator increases from L₁ to L₂ to L₃.

FIGS. 7D-7F provide further evidence of the relationship between various attributes. For example, FIG. 7D describes the relationship between displacement and volume in the presence of FIG. 7D further illustrates the relationship between volume and displacement, wherein increases in volume of working fluid provided to the planar actuator results in a monotonic increase in displacement or lengthening of the planar actuator. Similarly, FIG. 7E illustrates the relationship between volume and force, wherein the length of the planar actuator is held constant. In this example, as volume increases the force applied by the actuator decreases (assuming again, that length remains constant) as expected. Finally, FIG. 7F illustrates the relationship between the force displacement relationship with volume held constant. As expected, as volume increased, the rate of change of force with displacement, reflecting the actuator stiffness increased.

Although some of the data presented in FIGS. 7D-7F illustrates small hystereses and/or non-linearities, the data overall is in agreement with expectations. In addition, loads of 500 kPa, with no-load tests illustrating textile strain of over 40%.

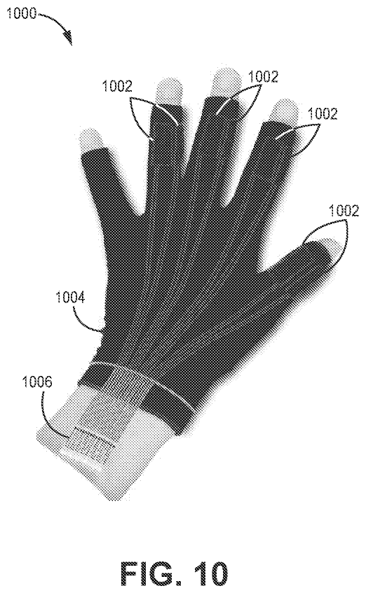

FIGS. 8A and 8B are schematic views of a soft actuator that incorporates one or more soft sensors according to some embodiments. In the embodiment shown in FIG. 8A, soft actuator 800 includes one or more hollow elastic fibers 802, elastic fabric 804, and circumferential constraint 806. A working fluid (e.g., hydraulic, pneumatic) is selectively applied to the one or more hollow elastic fibers 802 to selectively actuate the actuator as described above. That is, circumferential constraint 806 (e.g., stitching, side-stiches, cross-stitches) are provided in the fabric 804 to form one or more channels configured to receive the one or more hollow elastic fibers 802. Provision of a working fluid into the one or more hollow elastic fibers results in the actuation (e.g., lengthening) of soft actuator 800 in a direction parallel to the length of the one or more hollow elastic fibers 802. In the embodiment shown in FIG. 8A, in addition to the components utilized to selectively actuated the soft actuator, one or more strain sensors 808 are included in the soft actuator 800. In some embodiments, the strain sensors 808 are located adjacent one or more of the hollow elastic fibers 802 and extend along the length of the soft actuator. In some embodiments, an attribute (e.g., resistance) of the strain sensor changes in response to stretching or lengthening of the strain sensor 808. For example, in some embodiments strain sensor 808 may utilize a hollow tube (e.g., elastic) that is filled with a liquid metal. The conductivity (e.g., inverse of resistance) of the strain sensor 808 changes in response to a strain applied to the strain sensor (e.g., lengthening or stretching of the sensor). In some embodiments, the hollow tube comprising liquid metal is braided to selectively modify the sensitivity of the strain sensor. One of the benefits of including a strain sensor 808 as part of the soft actuator 800 is that it allows the strain data collected from the strain sensor 808 to be utilized in feedback to control the actuator of the soft actuator (i.e., detect the lengthening/movement of the actuator).

In the embodiment shown in FIG. 8B, soft actuator 820 includes one or more hollow elastic fibers 822, elastic fabric 824, and circumferential constraints (not explicitly shown in this view). A working fluid (e.g., hydraulic, pneumatic) is selectively applied to the one or more hollow elastic fibers 822 to selectively actuate the actuator as described above. In this embodiment, the soft actuator 820 is in a non-compression state when the working fluid is pressurized and in a compression state when the working fluid is de-pressurized (e.g., low working fluid pressure). For example, soft actuator 820 may be utilized as a compression sleeve or haptic sensor selectively providing pressure as required.

In the embodiment shown in FIG. 8B, in addition to the components utilized to selectively actuate the soft actuator, one or more pressure sensor 828 are included in the soft actuator 820. In some embodiments, the pressure sensors 828 are located in a grid pattern along the outer circumference of the soft actuator 820. In some embodiments, an attribute (e.g., resistance) of the strain sensor changes in response to pressure applied by the soft actuator 820 to an underlying surface (e.g., limb). As discussed above with respect to strain sensors, pressure sensor 828 work under the same principle, wherein the pressure sensors comprise one or more hollow tubes (e.g., elastic) filled with a liquid metal. The application of pressure to the pressure sensor changes the measured conductivity (e.g., resistance) associated with the pressure sensor. In some embodiments, the hollow tube comprising liquid metal is braided to selectively modify the sensitivity of the pressure sensor. One of the benefits of including a pressure sensor 828 as part of the soft actuator 800 is that it allows the pressure data collected from the pressure sensor 828 to be utilized in feedback to control the actuation of the soft actuator. For example, in embodiments in which the soft actuator 820 is utilized as a compression sleeve, the feedback can be utilized to apply a desired amount of compression (e.g., one size fits all compression sleeves).

For additional information regarding strain sensors is provided in PCT Appl. US2016/050769, titled “TACTILE SENSORS AND METHODS OF FABRICATING TACTILE SENSORS”, by Yon Visell and Bin Li, and PCT Application No. 2018/016214, titled “STRETCHABLE, CONDUCTIVE INTERCONNECT AND/OR SENSOR AND METHOD OF MAKING THE SAME” by Yon Visell and Do Thanh Nho, both of which are incorporated by reference herein.

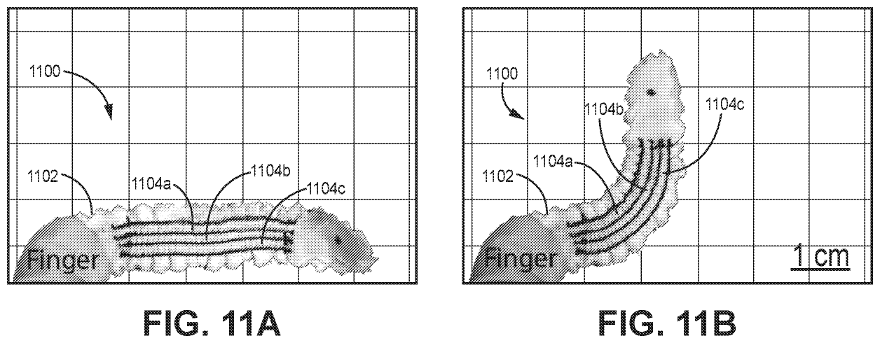

FIGS. 9A and 9B are photographs of strain sensors that may be incorporated into the soft actuator according to some embodiments. In particular, FIG. 9A shows an embodiment in which strain sensor 900 includes two microtubules 902a, 902b braided together with an inelastic component (e.g., sewing thread) 904 in order to increase the stiffness of the microtubules. As the strain sensor 900 is stretched, the braided portions apply force to one another, reducing the cross-sectional area of the liquid metal within the respective microtubules 902a, 902b and thereby reducing the conductivity of each (conversely, increasing the resistance of each). In addition, the result of braiding the microtubules with non-elastic thread 904 is that as the strain sensor 900 is stretched, more force is exerted at the turns of the microtubules 902a, 902b, resulting in a larger resistance change and therefore higher sensitivity.

Similarly, FIG. 9B shows an embodiment in which strain sensor 920 includes two microtubules 922a, 922b are braided with two elastic cords 924a, 924b. The elasticity of the elastic cords 924a, 924b decreases the strain applied on the microtubules 922a, 922b in response to stretching of the strain sensor 920, thereby decreasing the sensitivity of the strain sensor 920. The trade-off, however, is that strain sensor 920 is more flexible and provides a greater range of motion as compared with strain sensor 900.

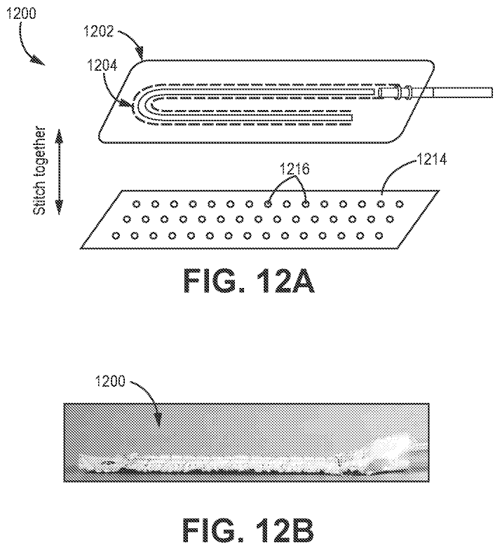

FIG. 10 is a perspective view of a garment 1000 (e.g., glove) that integrates a plurality of soft actuators 1002 to provide haptic feedback to a user. In some embodiments, the garment is utilized to provide haptic feedback to the hand for human-computer interaction or virtual reality. Garment 1000 includes a plurality of soft actuators 1002, a pair of tubes 1004 connected to each soft actuator, and a plurality of pumps 1006 connected to selectively provide a working fluid to each of the plurality of soft actuators 1002 via tubes 1004. In this way, one of the plurality of pumps 1006 may be utilized to selectively apply or remove a working fluid from one of the corresponding soft actuators 1002, thereby providing haptic feedback to the user. In some embodiments, the garment 1000 further includes one or more sensors (not shown) that provide feedback regarding position and/or movement of the user's fingers.

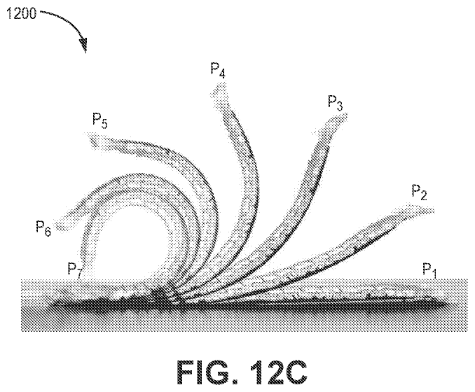

FIGS. 11a and 11b illustrates a multi-actuated Fluidic Fabric Muscle Sheet (FFMS) 1100 operating in a first state and a second state, respectively. The FFMS 1100 is comprised of a plurality of soft actuators 1104a, 1104b, and 1104c provided within a fabric 1102 according to some embodiments. Although not shown, each of the plurality of soft actuators 1104a-1104c is coupled via a tube to a pump (e.g., hydraulic pump, pneumatic pump, etc.) to selectively pressurize one or more of the soft actuators 1104a-1104c.

FIG. 11a illustrates FFMS 1100 in a state in which all three soft actuators 1104a-1104c are pressurized. The pressurization of each of the soft actuators 1104a, 1104c results in a lengthening of the FFMS 1100 in an axial direction. FIG. 11b illustrates the FFMS 1100 in a state in which one of the soft actuators 1104a is depressurized causing differential elongation in the respective soft actuators 1104a-1104c. As a result of the depressurization the soft actuator 1104a is shortened, inducing a large amplitude planar rotation of the FFMS 1100 as shown in FIG. 11b.

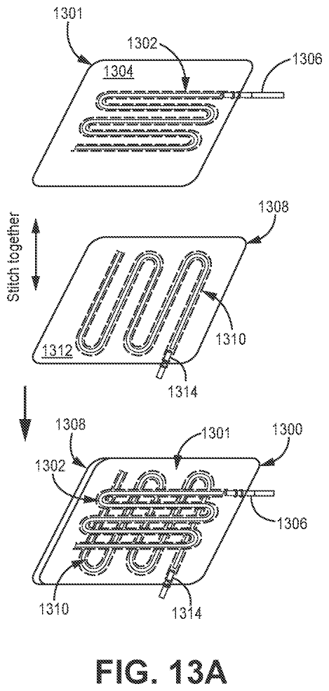

FIG. 12a-12c illustrate a FFMS 1200 capable of providing out-of-plane bending as a result of combination of a first soft actuator layer 1202 with a relatively stiff layer 1214. In the embodiment shown in FIG. 12a, a soft actuator layer 1202 includes one or more soft actuators 1204 (i.e., hollow elastic fiber combined with circumferential constraint) located in a first plane. The soft actuator layer 1202 is adhered or stitched to the relatively stiff, passive layer 1214. In some embodiments, the relatively stiff layer is a fiberglass sheet that includes a plurality of pre-patterned holes 1216 utilized to adhere the soft actuator layer 1202 to the stiff layer 1214. FIG. 12b is a side view of the FFMS 1200 in a first state (pressurized state). FIG. 12c is a side view of the FFMS 1200 at various pressure levels P1, P2, P3, P4, P5, and P6. In some embodiments, pressure P1 represents a maximum pressure level (e.g., approximately 724 kPa) and pressure P6 represents a minimum pressure level (e.g., 241 kPa). At a maximum pressure (e.g., 724 kPa) the one or more soft actuators 1204 are fully extended to provide a relatively flat surface. As the pressure decreases the length of the actuators shortens, but due to the attachment of the soft actuator layer 1202 to the relatively stiff, passive layer 1214 the FFMS 1200 achieves large amplitude bending. In some embodiments, the large amplitude bending exceeds 180°.

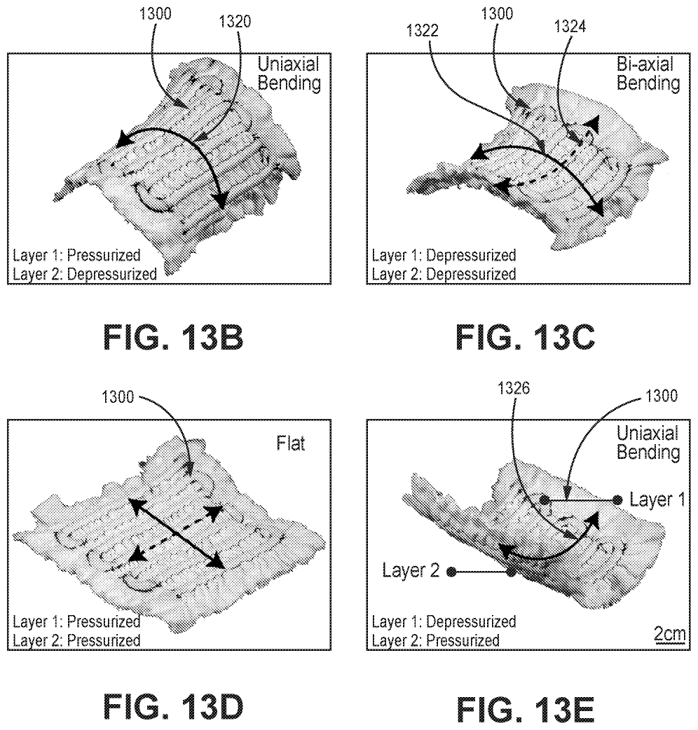

FIGS. 13a-13e illustrate a FFMS 1300 capable of biaxial bending. In some embodiments, FFMS 1300 utilizes first and second FFMS sheet actuators 1301 and 1308, aligned perpendicular to one another. Selective pressurization of the respective FFMS sheet actuators allows the FFMS to realize biaxial bending as shown in FIGS. 13b-13e.

In the embodiment shown in FIG. 13a, first FFMS sheet actuator 1301 includes one or more soft actuators 1302 (i.e., including hollow elastic fiber circumferentially constrained) located within fabric 1304 and aligned in a first direction, and second FFMS sheet actuator 1308 includes one or more soft actuators 1310 within fabric 1312 and aligned in a second direction perpendicular to the first direction. First FFMS sheet actuator 1301 includes an input port 1306 for receiving a working fluid (i.e., pneumatic, hydraulic, etc.), which would be connected to a pump (not shown) capable of selectively pressurizing the one or more soft actuators 1302. Likewise, second FFMS sheet actuator 1308 includes an input port 1314 for receiving a working fluid (i.e., pneumatic, hydraulic, etc.). In some embodiments, the first FFMS sheet actuator 1301 is stitched or otherwise adhered to the second FFMS sheet actuator 1308. In the embodiment shown in FIG. 13a the soft actuators 1302 associated with the first FFMS sheet actuator 1301 are aligned approximately perpendicular to the soft actuators 1310 associated with the second FFMS sheet actuator 1308. In other embodiments, the soft actuators associated with the first and second FFMS sheet actuators 1301, 1308 may be aligned at different angles relative to one another.

Based on the configuration shown in FIG. 13a, the FFMS 1300 may provide uniaxial bending along two different axes as well as bi-axial bending. For example, in the embodiment shown in FIG. 13b, uniaxial bending of FFMS 1300 is provided by pressurizing first FFMS sheet actuator 1301 and de-pressurizing second FFMS sheet actuator 1308. In response, FFMS 1300 bends around a single axis as indicated by arrow 1320. In the embodiment shown in FIG. 13c, bi-axial bending of FFMS 1300 is provided by de-pressurizing both first FFMS sheet actuator 1301 and second FFMS sheet actuator 1308. In response, FFMS 1300 bends around both a first axis as indicated by arrow 1322 as well as a second axis as indicated by arrow 1324. In the embodiment shown in FIG. 13d, FFMS 1300 is approximately flat as a result of pressurizing both first and second FFMS sheet actuators 1301 and 1308. In the embodiment shown in FIG. 13e, uniaxial bending of FFMS 1300 is provided by de-pressuring first FFMS sheet actuator 1301 and pressuring second FFMS sheet actuator 1308. In response, FFMS 1300 bends around a single axis as indicated by arrow 1326. However, as compared with the embodiment shown in FIG. 13b, FFMS 1300 bends around an axis perpendicular to the axis shown in FIG. 13b.

While the invention has been described with reference to an exemplary embodiment(s), it will be understood by those skilled in the art that various changes may be made and equivalents may be substituted for elements thereof without departing from the scope of the invention. In addition, many modifications may be made to adapt a particular situation or material to the teachings of the invention without departing from the essential scope thereof. Therefore, it is intended that the invention not be limited to the particular embodiment(s) disclosed, but that the invention will include all embodiments falling within the scope of the appended claims.

1. A method of fabricating a soft actuator, the method comprising:
stacking two or more layers of fabric adjacent to one another;
forming one or more channels between the stacked layers of fabric, wherein the channels provide a circumferential constraint;
placing one or more hollow elastic tubes within the one or more channels; and
connecting at least a first end of each of the one or more hollow elastic tubes to a delivery system capable of providing a working fluid to the one or more hollow elastic tubes.

2. The method of claim 1, further including:
sealing a second end of each of the one or more hollow elastic tubes.

3. The method of claim 1, further including:
connecting the second end of each of the one or more hollow elastic tubes to the delivery system capable of providing a working fluid to the one or more hollow elastic tubes.

4. The method of claim 1, wherein the two or more layers of fabric are non-stretch.

5. The method of claim 1, wherein the two or more layers of fabric are two-way stretch.

6. The method of claim 1, wherein forming the one or more channels between the stacked layers of fabric includes stitching the two or more layers of fabric together using a side-stitch to form the one or more channels.

7. The method of claim 6, wherein the stitching is provided in a direction transverse to a direction of lengthening, wherein the direction of lengthening is along a length of the hollow elastic tubes.

8. The method of claim 1, wherein forming the one or more channels between the stacked layers of fabric includes stitching the two or more layers of fabric together using a cross-stitch to form the one or more channels.

9. The method of claim 1, wherein forming the one or more channels between the stacked layers of fabric includes gluing the two or more layers of fabric together.

10. A selectively actuated textile comprising:
one or more pieces of fabric, wherein the one or more pieces of fabric include one or more channels formed within the one or more pieces of fabric and constrained in a circumferential direction; and
one or more first hollow elastic tubes positioned within the one or more channels and configured to receive a working fluid, wherein working fluid is selectively provided to or removed from the one or more first hollow elastic tubes to actuate the selectively actuated textile.

11. The selectively actuated textile of claim 10, wherein the fabric is elastic and allowed to stretch in response to the working fluid being selectively provided to the one or more first hollow elastic tubes.

12. The selectively actuated textile of claim 10, wherein the fabric is inelastic, wherein the fabric is bunched to allow the selectively actuated textile to lengthen in response to the working fluid being selectively provided to the one or more first hollow elastic tubes.

13. The selectively actuated textile of claim 10, wherein the one or more first hollow elastic tubes includes a first plurality of hollow elastic tubes positioned parallel to one another along a length of the selectively actuated textile.

14. The selectively actuated textile of claim 10, wherein the one or more channels is fabricated utilizing stitching along edges of each of the one or more channels.

15. The selectively actuated textile of claim 10, wherein the selectively actuated textile has a length, wherein the length changes in response to the working fluid being selectively provided to or removed from the one or more first hollow elastic tubes.

16. The selectively actuated textile of claim 15, wherein the length of the selectively actuated textile increases in response to working fluid being selectively provided to the one or more first hollow elastic tubes.

17. The selectively actuated textile of claim 10, wherein the selectively actuated textile is fixed on a first end, wherein a second end is actuated in response to the working fluid being selectively provided to or removed from the one or more first hollow elastic tubes.

18. The selectively actuated textile of claim 10, further including:
a strain sensor positioned in close proximity to the one or more hollow elastic tubes, wherein the strain sensor provides feedback regarding force or displacement applied by the selectively actuated textile.

19. The selectively actuated textile of claim 10, further including:
a passive layer of stiff material that is stitched to the actuated textile, wherein the passive layer enables out-of-plane bending.

20. The selectively actuated textile of claim 10, further including one or more second hollow elastic tubes oriented at an angle with respect to the one or more first hollow elastic tubes.

21. A haptic feedback garment comprising:
one or more soft actuators embedded within the haptic feedback garment, wherein each actuator includes a hollow elastic tube configured to receive a working fluid;
one or more tubes, wherein each tube is connected on a first end to an input associated with each of the soft actuators; and
one or more pumps, wherein each pump is configured to provide a working fluid to one of the one or more soft actuators via one of the one or more tubes, wherein application of the working fluid to the soft actuator provides a haptic response to a user wearing the haptic feedback garment.

22. The haptic feedback garment of claim 21, wherein the one or more soft actuators include a channel constrained in a circumferential direction, wherein the hollow elastic tube is positioned within the channel.

23. The haptic feedback garment of claim 21, wherein the haptic feedback garment is a glove having a plurality of fingers, wherein each finger includes one or more embedded soft actuators.

24. The haptic feedback garment of claim 21, wherein the haptic feedback garment is a sleeve or band and wherein each of the soft actuators is positioned circumferentially around the sleeve or band, wherein removal of fluid from the one or more soft actuators provides a compressive force to the sleeve or band.
